(19) United States
(12) Patent Application Publication
(10) Pub. No.: US 2015/0157224 A1
(43) Pub. Date: Jun. 11, 2015

(54) System and Method for Remotely Identifying and Characterizing Life Physiological Signs

(71) Applicant: SAMSUNG ELECTRONICS CO., LTD., Gyeonggi-do (KR)

(72) Inventors: Gideon CARMON, Haifa (IL); Dan SHIRRON, Givaat Ada (IL); Tsvi Chaim LEV, Tel Aviv (IL)

(73) Assignee: SAMSUNG ELECTRONICS CO., LTD., Gyeonggi-do (KR)

(21) Appl. No.: 14/097,336

(22) Filed: Dec. 5, 2013

(51) Int. Cl. A61B 5/024; A61B 5/00; A61B 5/16; A61B 5/08

(52) U.S. Cl. CPC A61B 5/02427; A61B 5/0816; A61B 5/7203; A61B 5/743; A61B 5/7282; A61B 5/02405; A61B 5/165; A61B 2576/023

(57) ABSTRACT

A system remotely determines living objects and their heartbeat rate or breath rate. The system has a laser source, a diffractive optics, a defocusing lens, a laser CCD, and a vibrations analyzer. The laser source is used for emitting a laser beam. The diffractive optics is used for dividing said laser beam to a grid of plurality of laser beams in which the living object is located. The defocusing lens is used for receiving pluralities of laser beams as reflected from objects within the area of interest, and for defocusing said reflected beams, thereby to produce defocused speckles on the laser CCD. The vibrations analyzer is used for analyzing vibrations within said displayed speckles, and for determining therefrom a vibrations rate of heartbeat or breath respectively of the living object

### FIELD OF THE INVENTION

The field of the invention relates to the field of determination and measurement of life physiological signs. More particularly, the invention relates to a method and system for remotely determining whether there are objects in a scene with life physiological signs that generate micro-movements such as heart beats or breathing, or determining the heartbeat and breathing rate of one or more persons that are located within a specific location.

### BACKGROUND OF THE INVENTION

Heart beat and breathing are typical to all leaving creatures. Typically, a human being breathes at a rate ranging from 30-60 breaths per minute (for a newborn baby) to 12-16 breaths per minute for an adult. A normal heart beat rate for an adult ranges between 60-100 times per minute at a resting condition.

It is well known that the breathing rate and the heart beat depend on the health, physiological, rhythmic conditions, but also on a frame of mind of the person involved. Therefore, in many cases the breathing rate and the heartbeat are measured in order to determine the condition of the person involved, in terms of health, mood, physiological condition, etc. For example, the heartbeat of patients is regularly checked in hospitals as an essential parameter which relates to the patient's health condition.

Another example is the so-called “baby sense” monitor, which is an instrument for monitoring and ensuring appropriate breathing of babies. This instrument is typically placed under the bassinette or crib mattress of the baby. It comprises a sensor for sensing the baby breathing and movements.

In another aspect, it is well known that a state of mind of a person affects his breathing rate and his heartbeat. For example, a polygraph instrument uses electrodes that are attached to the body of the person, and this instrument bases its conclusion, among other parameters, also on the breathing rate and on the heartbeat of the person involved.

A typical instrument for checking the heartbeat of a person comprises one or more electrodes that are directly attached to the human body. Moreover, each of such heartbeat measuring instruments is typically designed for an individual person, and cannot be used for a simultaneous measurement of the heartbeat of plurality of persons.

In still another aspect, the security at various locations, such as airports, malls, stores, etc. is based on an inspection and check of behavior and mood of a mass of people, thereby to conclude whether any of them poses a potential risk. For this purpose, security personnel are instructed to inspect the scene, and to investigate suspicious persons, when a necessity arisen, in order to evaluate their potential risk. Obviously, this investigation (when performed) and check is based on a subjective evaluation, as it mostly depends on an evaluation and conclusion of the security person involved. In this respect, however, a state of mind of a human being typically affects his breathing rate and also his heartbeat. Still, these parameters are presently not considered for these security purposes, particularly as they require direct attachment of electrodes to the body of each subject, which is in most cases innocent. Such a direct attachment of electrodes to the body of the subjects is obviously impractical, resulting in a less and mostly subjective conclusion with respect to the risk.

It is therefore an object of the present invention to provide a method and system for remotely determining the breath rate and heartbeat rate of a patient.

It is another object of the present invention to provide said method and system for remotely determining the breath rate and heartbeat rate, in a manner which is operated by a single instrument to plurality of persons simultaneously.

It is still another object of the present invention to provide a method and system for remotely inspecting the health condition of human beings.

It is still another object of the present invention to use said method and system for remotely concluding with respect to a state of mind of a human being.

It is still another object of the present invention to use said method and system for remotely determining a rhythmic condition of a human being.

It is still another object of the present invention to use said method and system for remotely determining the number of persons located in a specific scene and to identify their position.

Other objects and advantages of the invention will become apparent as the description proceeds.

### SUMMARY OF THE INVENTION

The invention relates to a system for remotely determining the heartbeat rate or breath rate of a person, which comprises: (a) a laser source for emitting a laser beam; (b) a diffractive optics for dividing said laser beam to a grid of plurality of laser beams that are propagated toward a space of interest in which said person is located; (c) a defocusing lens for receiving pluralities of laser beams as reflected from objects within the area of interest, and for defocusing said reflected beams, thereby to produce defocused speckles on a laser CCD; (d) a laser CCD which is sensitive in the laser range, for displaying said produced speckles; and (e) a vibrations analyzer for analyzing vibrations within said displayed speckles, and for determining therefrom a vibrations rate of heartbeat or breath respectively of said person.

Preferably, said heartbeat or breath rate conform to the respective rate of the speckles vibrations.

Preferably, one of more range filters are used by said analyzer in order to pass only a relevant range of speckle vibrations, and to eliminate irrelevant speckle vibrations that are caused by irrelevant objects or irrelevant persons movements.

Preferably, respective levels of the determined heartbeat or breath vibrations are converted to color levels, and overlaid on a visual display of each respective person which is located within the space of interest. A system according to claim 1, wherein respective levels of the determined heartbeat or breath vibrations are displayed adjacent to each respective person which is located within the space of interest.

Preferably, the system is used to determine variations in heartbeat rate or breath rate, that are caused due to a health condition, a mood, or a physiological condition of a person.

Preferably, the system is used is used to merely detect existence of human beings within a scene.

Preferably, the system is used is used to detect existence of human beings within a scene, and their locations.

Preferably, the system is used to detect the number of human beings within a scene.

The invention also relates to a method for remotely determining the heartbeat rate or breath rate of a person, which comprises: (a) emitting a laser ray from a laser source; (b) dividing said laser beam to a grid of plurality of laser beams, and propagating said plurality of laser beams toward a space of interest in which said person is located; (c) defocusing the laser beams as reflected from objects within the area of interest, thereby to produce respective defocused speckles on a laser CCD; and (d) analyzing vibrations within said displayed speckles, and determining therefrom a vibrations rate of heartbeat or breath respectively of said person.

Preferably, irrelevant speckle vibration rates are eliminated and ignored in said analyzing step.

### BRIEF DESCRIPTION OF THE DRAWINGS

In the drawings:

FIG. 1 schematically describes the speckles phenomenon, which is used by the present invention;

FIG. 2a-2c describe the speckles phenomenon, as imaged by a system according to the present invention;

FIG. 3 shows a block diagram of an embodiment according to the present invention; and

FIG. 4 is a photograph showing display of exemplary results as obtained by the system of the present invention.

### DETAILED DESCRIPTION OF EMBODIMENTS OF THE INVENTION

As noted, an object of the present invention is to remotely determine in a simultaneous manner the heartbeat rate and the breathing rate of plurality of persons. Another object of the present invention is to use said method and system for remotely determining the number of persons that are located in a specific scene and to identify their position.

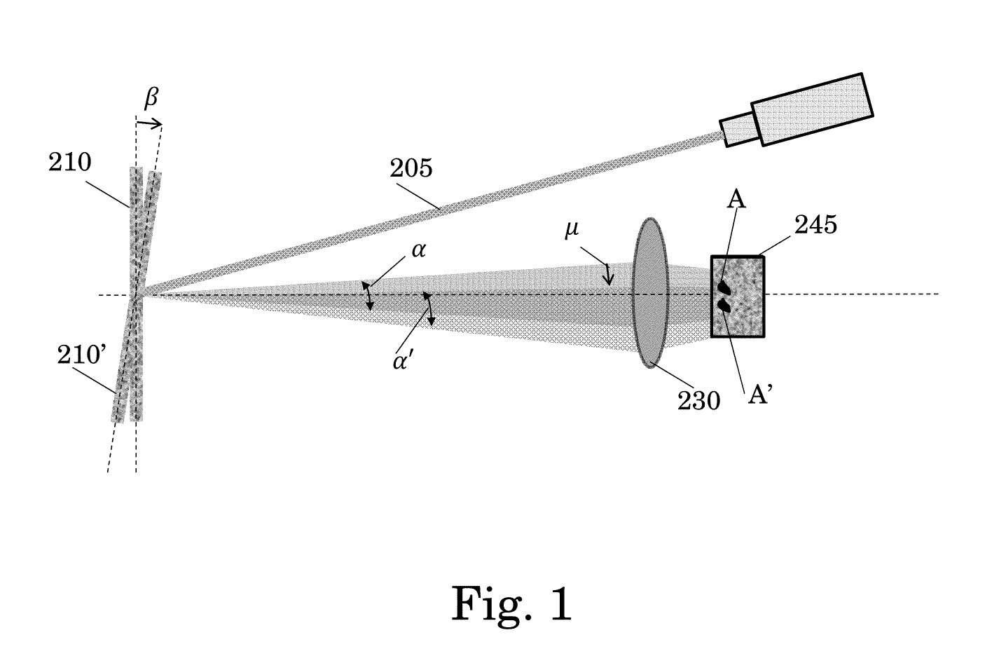

FIG. 1 illustrates a phenomenon known in the art which occurs when a coherent laser beam 205 is applied to a somewhat rough surface 210. When laser beam 205 impinges on surface 210, the laser beam is reflected toward lens 230, as shown by the rays spanning the angle α. These rays, upon passing through lens 230, are directed toward CCD 245, and produce a speckle type image A at a specific location over CCD 245. The speckle type image A which is produced on CCD 245 is not homogeneous (i.e., the image has some random pattern), in view of the roughness of surface 210. Clearly, each surface has some level of roughness, and this roughness is reflected on the CCD 245. When surface 210 is tilted by an angle β relative to its original vertical state, i.e., to an orientation 210′, the reflected rays are also displaced by a displacement μ, to an orientation as spanning the angle α′. As a result, the speckle-type virtual image A is also vertically displaced over CCD 245 to a location A′. According to the present invention, the lens 230 is a defocusing lens which intensifies said displacement of image A, and CCD 245 is an array which is sensitive to light in the wavelength of the laser beam. A more detailed description of this phenomenon is described in M. Francon, “Laser Speckle and Applications in Optics”, pages 100-102, Academic Press, 1979, and in R. Jones & C. Wykes, “Holographic and Speckle Interferometry”, Second Edition, Cambridge Studies in Modern Optics, Cambridge University Press, pages 135-139.

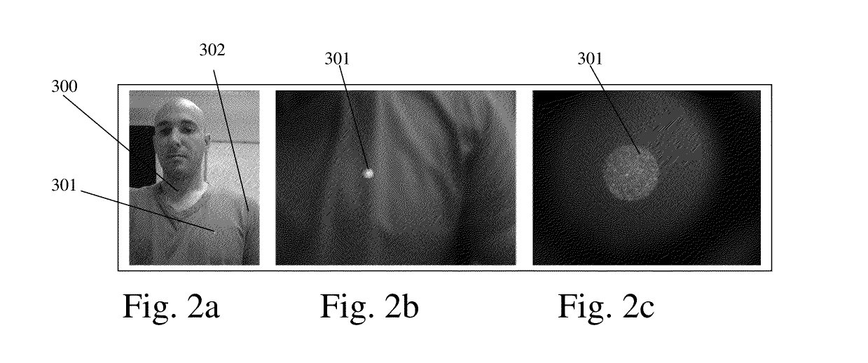

FIG. 2a shows a spot 301 which is formed by a coherent laser beam which is impinged on a shirt 302 of a person. FIG. 2b shows the spot 301 in somewhat enlarged scale, and FIG. 2c shows a same spot as seen when a defocusing lens (such as lens 230 of FIG. 1) is provided in front of the CCD 245 which senses the image of the spot as produced by the laser light rays that are impinged on it. A close look into the defocused spot 301 of FIG. 2c shows that the spot is in fact formed from many “speckles” that are randomly disposed within area of the spot 301. The speckles are formed due to the roughness of shirt 302. Furthermore, it can be seen that these speckles are not stationary within the area of the spot 301, but in fact their locations are displaced respectively in a vibratory manner within the area of spot 301. The vibratory-type displacement of the speckles is resulted from the micro-movement of shirt 302, which is in turn caused by the breathing and heartbeat of person 300. In such a manner a micro-movement of an object, such as shirt 302 that is dressed by person 300, can be determined by means of inspecting the micro movement the speckles within spot 301, as provided over a CCD 245. The amplitude of said speckles displacement vibrations within spot 301 is relative to the micro-movement of said shirt due to the breathing and heart bit. The frequency of vibration of the speckles within spot 301 is in fact a combination between the frequency of breathing of person 300 and his frequency of heartbeat, respectively. It has been found by the inventors that an image analysis of said speckle vibrations, as continuously provided on said CCD 245, can reveal the heartbeat rate and the breathing rate of the person.

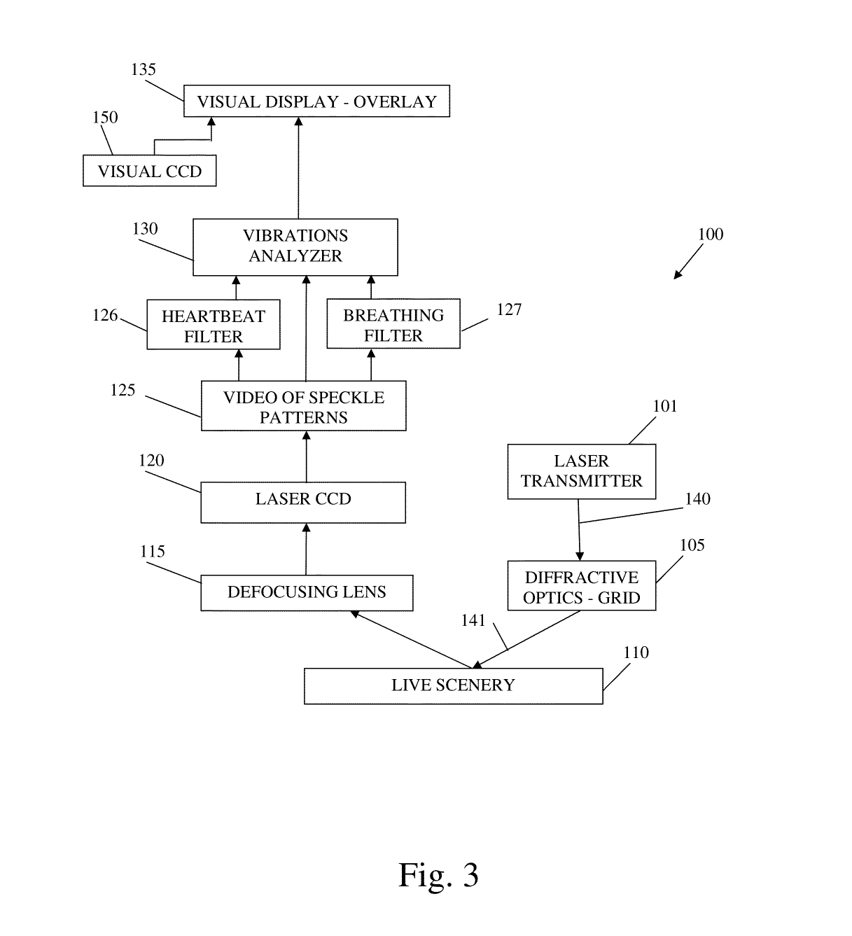

The system of FIG. 1 comprises a single laser beam, which is suitable for determination of a heartbeat and breathing of a single person, assuming that the person is relatively stationary. FIG. 3 shows in a block diagram form a structure of a system which is suitable for determination of heartbeat and breathing of several persons located within wide scenery, such as a hospital room with several patients. A single laser transmitter 101 produces a single laser beam 140. The beam 140 is passed through a diffractive optics, which divides the beam 140 into a grid of many beams 141. The plurality of beams 141 are transmitted into the space of the room, and impinge on all objects within the room, including those people located within the room. A CCD 120, which is sensitive to the wavelength of the laser beam, captures video of the entire room scenery through a defocusing lens 115. A video stream 125 of the entire room, including the plurality of speckle images is produced by CCD 120. As noted, a laser beam which impinges on a stationary surface produces a spot with stationary speckles. When a laser beam impinges on a shirt of a person, it produces on the CCD 120 a spot with vibrating speckles. Clearly, there are various types of movements within the room. For example, each person within the room moves in a manner which is independent from the micro-movement of his heartbeat and breathing. Furthermore, an air conditioner within the room may also vibrate in some high frequency. However, while knowing the typical range of the heartbeat frequency and the typical range of the breathing frequency, use of appropriate range filters can eliminate irrelevant components from the signal, thereby enabling analysis of only the heartbeat rate or only the breathing rate at each specific period. More specifically, a breathing range filter 127 and a heartbeat range filter 126 are used, enabling an image analyzer (image processor) to “inspect” said filtered vibrations respectively, thereby to determine locations within the scenery where a live person is located (i.e., locations where vibrations that are typical to breath or heartbeat respectively are detected). This determination becomes possible in view of the isolation by said filters 126 and 127 of the relevant heartbeat or breathing frequency ranges respectively, from other displacements that may occur in the scene. As said, vibrations in other frequencies or random movements are eliminated and ignored by use of said filters. Moreover, the frequencies of vibrations (rates) can be determined, by analyzing the video images.

In still another embodiment, a conventional CCD camera 150, which is sensitive to the visual light range, is used to capture a conventional visual scene of the same space of interest. The two outputs, i.e., from the visual CCD camera 150, and from the vibrations analyzer 130 are then combined and overlaid, thereby to produce an overlay visual display 135. In one example, the determined vibration rates at each location may be converted to color. For example, a lowest vibration frequency of the speckles may be converted to levels of a yellow color, higher vibrations frequencies to levels orange, and highest vibrations frequencies to levels red. In such a manner, after spatially overlaying of the determined vibrations, as converted to levels of colors, over the visual display, the level rates of heartbeat or breathing rates are assigned to the respective persons, and displayed.

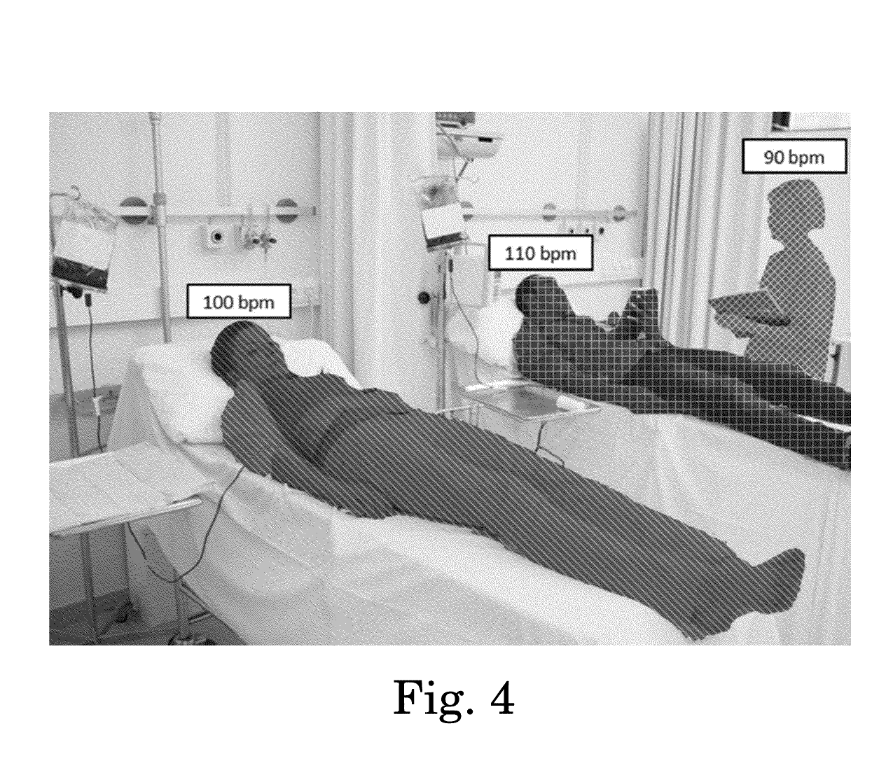

FIG. 4 shows a hospital room in which several patients stay. Preferably, the patients are colored relative to their level of heart beat or breathing rate. Therefore, by looking at the intensity of the color, it can be seen that the patient at the right bed has a higher heartbeat rate than the other persons in the room. This high heartbeat rate, as determined by the system of the present invention, is indicated by coloring the person at the image by red color. The actual rate of this patient heartbeat is in fact 110 bpm. The level of the heartbeat of the patient (woman) at the left bed is somewhat lower, and this rate is indicated by an orange color at the image (the actual heartbeat is 100 bpm). The heartbeat rate of the woman (nurse) which is standing at the right is the lowest, and it is preferably indicated by yellow color—her actual heartbeat is 90 bpm. It can also be easily seen that the operative air conditioner, which most probably vibrates at a much higher rate than the breathing and the heartbeat frequencies of the persons at the room, is not marked by any color, as its speckles vibration is out of the ranges of the breathing and heartbeat filters 126 and 127. Alternatively, instead of using a color level for the vibrations indications, numeral indications may be provided adjacent each person, as also shown in FIG. 4.

In such a manner, when using a converter which converts from an actual vibration rate to color, a person with a too high heartbeat rate can be easily observed in real time, no matter what is the reason for such a high heartbeat (either due to a health problem, or due to his state of mind, or otherwise). Therefore, the system as described may be used in a hospital for determining simultaneously the health conditions of several patients. The system of the invention may alternatively be used in a secured area, for example, an airport, where a person who poses a security risk can be easily detected based on his abnormal rate of heartbeat of breath.

The system of the present invention, as described, may also be used to merely detect existence of human beings within a scene (by detecting breathing and heartbeat). In another embodiment, the system of the invention may be used to also detect the locations of said persons within the scene. In still another embodiment, the system of the invention may be used to detect the number of people within a scene.

It should be noted that in order to avoid damage to the vision of the people, the system uses a laser class 1.

While some embodiments of the invention have been described by way of illustration, it will be apparent that the invention can be carried into practice with many modifications, variations and adaptations, and with the use of the numerous equivalents or alternative solutions that are within the scope of persons skilled in the art, without departing from the spirit of the invention or exceeding the scope of the claims.

1. A system for remotely determining living objects and their heartbeat rate or breath rate, which comprises:
a. a laser source for emitting a laser beam;
b. a diffractive optics for dividing said laser beam to a grid of plurality of laser beams that are propagated toward a space of interest in which said living object is located;
c. a defocusing lens for receiving pluralities of laser beams as reflected from objects within the area of interest, and for defocusing said reflected beams, thereby to produce defocused speckles on a laser CCD;
d. a laser CCD which is sensitive in the laser range, for displaying said produced speckles; and
e. a vibrations analyzer for analyzing vibrations within said displayed speckles, and for determining therefrom a vibrations rate of heartbeat or breath respectively of said living object.

2. A system according to claim 1, wherein said heartbeat or breath rate conform to the respective rate of the speckles vibrations.

3. A system according to claim 1, wherein one of more range filters are used by said analyzer in order to pass only a relevant range of speckle vibrations, and to eliminate irrelevant speckle vibrations that are caused by irrelevant objects or irrelevant living objects movements.

4. A system according to claim 1, wherein respective levels of the determined heartbeat or breath vibrations are converted to color levels, and overlaid on a visual display of each respective living object which is located within the space of interest.

5. A system according to claim 1, wherein respective levels of the determined heartbeat or breath vibrations are displayed adjacent to each respective living object which is located within the space of interest.

6. System according to claim 1, which is used to determine variations in heartbeat rate or breath rate, that are caused due to a health condition, a state of mood, or a physiological condition of a living object.

7. A system according to claim 1, wherein a spatial analysis of each pixel of said vibrations within said displayed speckles is used to spatially determine and differentiate between living objects, and to derive their respective physiological properties.

8. System according to claim 1, which is used to merely detect existence of human beings within a scene.

9. System according to claim 8, which is used to detect existence of human beings within a scene, and their locations.

10. System according to claim 8, which is used to detect the number of human beings within a scene.

11. A method for remotely determining the heartbeat rate or breath rate of a person, which comprises:
a. emitting a laser ray from a laser source;
b. dividing said laser beam to a grid of plurality of laser beams, and propagating said plurality of laser beams toward a space of interest in which said person is located;
c. defocusing the laser beams as reflected from objects within the area of interest, thereby to produce respective defocused speckles on a laser CCD; and
d. analyzing vibrations within said displayed speckles, and determining therefrom a vibrations rate of heartbeat or breath respectively of said person.

12. A method according to claim 11, wherein irrelevant speckle vibration rates are eliminated and ignored in said analyzing step.
